SEQUENCE LISTING (15 sequences)

SEQ ID NO: 1 - protein, 66 aa, Artificial Sequence
  Description of Artificial Sequence Synthetic polypeptide
CXXXXCXXXXXXXXXXXXXXXXXXXWHXXXXCFXCXXXXCXXXXXXXXXXXXXXXXXXXXCXXXXC

SEQ ID NO: 2 - protein, 5 aa, Artificial Sequence
  Description of Artificial Sequence Synthetic peptide
WSXWS

SEQ ID NO: 3 - protein, 19 aa, Pseudomonas stutzeri
RLIDPALLAHPNTLFTPHI

SEQ ID NO: 4 - protein, 10 aa, Pseudomonas stutzeri
SGEFQGWQPQ

SEQ ID NO: 5 - protein, 25 aa, Pseudomonas stutzeri
VFEMEDWARADRPRLIDPALLAHPN

SEQ ID NO: 6 - protein, 336 aa, Pseudomonas stutzeri
MLPKLVITHRVHDEILQLLAPHCELMTNQTDSTLTREEILRRCRDAQAMMAFMPDRVDADFLQACPELRVVGCALKGFDNFDVDACTARGVWLTFVPDLLTVPTAELAIGLAVGLGRHLRAADAFVRSGEFQGWQPQFYGTGLDNATVGILGMGAIGLAMADRLQGWGATLQYHEAKALDTQTEQRLGLRQVACSELFASSDFILLALPLNADTQHLVNAELLALVRPGALLVNPCRGSVVDEAAVLAALERGQLGGYAADVFEMEDWARADRPRLIDPALLAHPNTLFTPHIGSAVRAVRLEIERCAAQNIIQVLAGARPINAANRLPKAEPAAC

SEQ ID NO: 7 - DNA, 1011 bp, Pseudomonas stutzeri
atgctgccgaaactcgttataactcaccgagtacacgatgagatcctgcaactgctggcgccacattgcgagctgatgaccaaccagaccgacagcacgctgacgcgcgaggaaattctgcgccgctgtcgcgatgctcaggcgatgatggcgttcatgcccgatcgggtcgatgcagactttcttcaagcctgccctgagctgcgtgtagtcggctgcgcgctcaagggcttcgacaatttcgatgtggacgcctgtactgcccgcggggtctggctgaccttcgtgcctgatctgttgacggtcccgactgccgagctggcgatcggactggcggtggggctggggcggcatctgcgggcagcagatgcgttcgtccgctctggcgagttccagggctggcaaccacagttctacggcacggggctggataacgctacggtcggcatccttggcatgggcgccatcggactggccatggctgatcgcttgcagggatggggcgcgaccctgcagtaccacgaggcgaaggctctggatacacaaaccgagcaacggctcggcctgcgccaggtggcgtgcagcgaactcttcgccagctcggacttcatcctgctggcgcttcccttgaatgccgatacccagcatctggtcaacgccgagctgcttgccctcgtacggccgggcgctctgcttgtaaacccctgtcgtggttcggtagtggatgaagccgccgtgctcgcggcgcttgagcgaggccagctcggcgggtatgcggcggatgtattcgaaatggaagactgggctcgcgcggaccggccgcggctgatcgatcctgcgctgctcgcgcatccgaatacgctgttcactccgcacatagggtcggcagtgcgcgcggtgcgcctggagattgaacgttgtgcagcgcagaacatcatccaggtattggcaggtgcgcgcccaatcaacgctgcgaaccgtctgcccaaggccgagcctgccgcatgttga

SEQ ID NO: 8 - protein, 281 aa, Pseudomonas stutzeri
MLPKLVITHRVHDEILQLLAPHCELMTNQTDSTLTREEILRRCRDAQAMMAFMPDRVDADFLQACPELRVVGCALKGFDNFDVDACTARGVWLTFVPDLLTVPTAELAIGLAVGLGRHLRAADAFVRSGEFQGWQPQFYGTGLDNATVGILGMGAIGLAMADRLQGWGATLQYHEAKALDTQTEQRLGLRQVACSELFASSDFILLALPLNADTQHLVNAELLALVRPGALLVNPCRGSVVDEAAVLAALERGQLGGYAADVFEMEDWARADRPRLIDPAL

SEQ ID NO: 9 - DNA, 843 bp, Pseudomonas stutzeri
atgctgccgaaactcgttataactcaccgagtacacgatgagatcctgcaactgctggcgccacattgcgagctgatgaccaaccagaccgacagcacgctgacgcgcgaggaaattctgcgccgctgtcgcgatgctcaggcgatgatggcgttcatgcccgatcgggtcgatgcagactttcttcaagcctgccctgagctgcgtgtagtcggctgcgcgctcaagggcttcgacaatttcgatgtggacgcctgtactgcccgcggggtctggctgaccttcgtgcctgatctgttgacggtcccgactgccgagctggcgatcggactggcggtggggctggggcggcatctgcgggcagcagatgcgttcgtccgctctggcgagttccagggctggcaaccacagttctacggcacggggctggataacgctacggtcggcatccttggcatgggcgccatcggactggccatggctgatcgcttgcagggatggggcgcgaccctgcagtaccacgaggcgaaggctctggatacacaaaccgagcaacggctcggcctgcgccaggtggcgtgcagcgaactcttcgccagctcggacttcatcctgctggcgcttcccttgaatgccgatacccagcatctggtcaacgccgagctgcttgccctcgtacggccgggcgctctgcttgtaaacccctgtcgtggttcggtagtggatgaagccgccgtgctcgcggcgcttgagcgaggccagctcggcgggtatgcggcggatgtattcgaaatggaagactgggctcgcgcggaccggccgcggctgatcgatcctgcgctg

SEQ ID NO: 10 - protein, 55 aa, Pseudomonas stutzeri
LAHPNTLFTPHIGSAVRAVRLEIERCAAQNIIQVLAGARPINAANRLPKAEPAAC

SEQ ID NO: 11 - DNA, 168 bp, Pseudomonas stutzeri
ctcgcgcatccgaatacgctgttcactccgcacatagggtcggcagtgcgcgcggtgcgcctggagattgaacgttgtgcagcgcagaacatcatccaggtattggcaggtgcgcgcccaatcaacgctgcgaaccgtctgcccaaggccgagcctgccgcatgttga

SEQ ID NO: 12 - protein, 29 aa, Pseudomonas stutzeri
ALKKELQANKKELAQLKWELQALKKELAQ

SEQ ID NO: 13 - DNA, 90 bp, Pseudomonas stutzeri
gctttaaaaaaagaattacaagctaataaaaaagaattagctcaattaaaatgggaattacaagctttaaaaaaagaattagctcaatga

SEQ ID NO: 14 - protein, 31 aa, Pseudomonas stutzeri
MEQLEKKLQALEKKLAQLEWKNQALEKKLAQ

SEQ ID NO: 15 - DNA, 93 bp, Pseudomonas stutzeri
atggaacaattagaaaaaaaattacaagctttagaaaaaaaattagctcaattagaatggaaaaatcaagctttagaaaaaaaattagctcaa

(19) United States
(12) Patent Application Publication
(10) Pub. No.: US 2017/0204401 A1
(43) Pub. Date: Jul. 20, 2017

(54) REDUCING HORIZONTAL GENE TRANSFER OF FUNCTIONAL PROTEINS

(71) Applicant: Novogy, Inc., Cambridge, MA (US)

(72) Inventors: Elena E. Brevnova, Belmont, MA (US); Emily H. Greenhagen, Melrose, MA (US); Arthur J. Shaw, IV, Belmont, MA (US); Colin R. South, Lexington, MA (US)

(73) Assignee: Novogy, Inc., Cambridge, MA (US)

(21) Appl. No.: 15/122,676

(22) PCT Filed: Mar. 3, 2015
(86) PCT No.: PCT/US2015/018410

§ 371 Date: Aug. 31, 2016

Related U.S. Application Data
(60) Provisional application No. 61/947,051, filed on Mar. 3, 2014.

(51) Int. Cl. C12N 15/10; C12N 15/65; C12N 15/52

(52) U.S. Cl. CPC C12N 15/1055; C12N 15/52; C12N 15/65

(57) ABSTRACT

Disclosed are methods and compositions for reducing horizontal gene transfer of functional proteins. In some embodiments, the risk of horizontal gene transfer of a functional protein is reduced by separately encoding domains of a protein on at least two spatially distinct nucleic acid sequences, where each individual domain alone is non-functional, but co-expression of the encoded domains results in their association to form a functional protein.

### RELATED APPLICATION

This application claims the benefit of U.S. Provisional Patent Application Ser. No. 61/947,051, filed Mar. 3, 2014, which application is hereby incorporated by reference.

### BACKGROUND

Horizontal gene transfer (HGT) is the transmission of genes from one organism to another through a mechanism other than sexual or asexual reproduction. Numerous processes can result in HGT. For example, in some instances viruses or retroviruses will package and transfer between cells cellular genetic material along with or instead of viral sequences. Genetic material will also pass between prokaryotes through endogenous bacterial processes, including bacterial transformation (the uptake and expression of environmental DNA) and conjugation (the transfer of DNA between bacteria that are in contact with each other).

HGT is a key mechanism through which microorganisms spread genes that convey a survival or growth advantage, such as antibiotic resistance genes, and virulence factors. For example, genes responsible for antibiotic resistance in one species of bacteria can be transferred to other species of bacteria through the various HGT mechanisms described above, resulting in the formation of new antibiotic resistant bacterial strains.

The risk of HGT is a concern when genes that convey a survival advantage are artificially engineered into organisms. Specifically, the HGT mediated spread of selectable markers (genes that facilitate cell survival under certain cell culture conditions) from engineered microorganisms to native microorganisms can be problematic in many fields, including medicine, agriculture and research. For example, selectable markers, including antibiotic resistance genes, are commonly used in microbiology to facilitate the selection of rare cells containing desired genetic material from a population of otherwise similar cells. Unfortunately, a researcher using an antibiotic resistance gene as a selectable marker in an experimental microorganism runs the risk that the gene will be horizontally transferred to a virulent microorganism, rendering the virulent microorganism resistant to the antibiotic and more difficult to treat medically. Even when the recipient microorganism is not virulent, spread of the antibiotic resistance gene renders the antibiotic less effective, increasing the risk of cell culture contamination. Similarly, HGT can result in herbicide resistance genes in genetically modified crops being transferred to invasive plant species, rendering the invasive plants herbicide resistant and more difficult to control.

Thus, there is a substantial need for new compositions and methods that reduce the risk of horizontal gene transfer of functional selectable markers from genetically modified organisms to the environment.

### SUMMARY

Provided herein are methods and compositions for reducing horizontal gene transfer of functional proteins. In some embodiments, the risk of horizontal gene transfer is reduced by separately encoding domains of a protein (e.g., a dominant selectable marker) on at least two spatially distinct nucleic acid sequences. The separated domains are engineered such that each individual domain, on its own, is non-functional, but co-expression of the domains together causes them to associate to form a functional protein. Because transfer of the functional protein would require the transfer of each of the multiple, spatially distinct nucleic acid sequences encoding the individual domains of the protein to a single cell, the risk of horizontal transfer of the functional protein is substantially reduced.

In some aspects, provided herein is a method of generating a cell that expresses a functional selectable marker, the method including introducing into a cell at least two different polynucleotides encoding different domains of a dominant selectable marker. In some embodiments, exactly two different polynucleotides are introduced. In some embodiments, the different domains of the dominant selectable marker associate to form a functional dominant selectable marker. In some embodiments, no individual polynucleotide encodes the functional dominant selectable marker. In some embodiments, the polynucleotides are introduced to the cell in vectors. In certain embodiments, the polynucleotides are plasmids, fragments of plasmids, or linearized plasmids. In some embodiments, the different polynucleotides integrate into different positions in the genome of the cell. In some embodiments, the polynucleotides are targeted to integrate into different positions in the genome of the cell (e.g., through homologous recombination). In some embodiments, the polynucleotides integrate into random positions in the genome of the cell.

In some aspects, provided herein is a method of generating a cell that expresses a functional dominant selectable marker, the method including introducing into a cell that expresses a first domain of a dominant selectable marker, a polynucleotide encoding a second domain of the dominant selectable marker. In some embodiments, the first domain and the second domain associate to form a functional dominant selectable marker. In some embodiments, the polynucleotide does not encode the functional dominant selectable marker; and the cell did not express the functional dominant selectable marker before transfection. In some embodiments, the polynucleotide is introduced to the cell in a vector. In certain embodiments, the polynucleotide is a plasmid, a fragment of a plasmid, or a linearized plasmid. In some embodiments, the polynucleotides integrates into the genome of the cell. In some embodiments, the polynucleotides are targeted to integrate into different positions in the genome of the cell (e.g., through homologous recombination). In some embodiments, the polynucleotides integrate into random positions in the genome of the cell.

In certain aspects, provided herein is a cell comprising at least two nucleic acid sequences located at different positions in the genome of the cell and encoding different domains of a dominant selectable marker. In some embodiments, the different domains of the dominant selectable marker associate to form a functional selectable marker. In some embodiments, no individual nucleic acid sequence encodes the functional dominant selectable marker. In some embodiments, the cell comprises exactly two nucleic acid sequences, each encoding a different domain of a dominant selectable marker, wherein the two different domains encoded by the polynucleotides associate in the cell to form the functional dominant selectable marker.

In some aspects, provided herein is a kit comprising at least two different polynucleotides encoding different domains of a dominant selectable marker. In some embodiments, the different domains of the dominant selectable marker are capable of associating to form a complete dominant selectable marker. In some embodiments, no individual polynucleotide encodes the entire dominant selectable marker. In some embodiments, the polynucleotides are in vectors. In some embodiments, the polynucleotides are plasmids, fragments of plasmids, or linearized plasmids. In some embodiments, the kit comprises exactly two different polynucleotides. In some embodiments, the kit further comprises a cell.

In some embodiments, the dominant selectable marker is a drug resistance marker. In some embodiments, the drug resistance marker confers resistance to a drug selected from the group consisting of Amphotericin B, Candicidin, Filipin, Hamycin, Natamycin, Nystatin, Rimocidin, Bifonazole, Butoconazole, Clotrimazole, Econazole, Fenticonazole, Isoconazole, Ketoconazole, Luliconazole, Miconazole, Omoconazole, Oxiconazole, Sertaconazole, Sulconazole, Tioconazole, Albaconazole, Fluconazole, Isavuconazole, Itraconazole, Posaconazole, Ravuconazole, Terconazole, Voriconazole, Abafungin, Amorolfin, Butenafine, Naftifine, Terbinafine, Anidulafungin, Caspofungin, Micafungin, Benzoic acid, Ciclopirox, Flucytosine, 5-fluorocytosine, Griseofulvin, Haloprogin, Polygodial, Tolnaftate, Crystal violet, Amikacin, Gentamicin, Kanamycin, Neomycin, Netilmicin, Tobramycin, Paromomycin, Spectinomycin, Geldanamycin, Herbimycin, Rifaximin, Streptomycin, Loracarbef, Ertapenem, Doripenem, Imipenem, Meropenem, Cefadroxil, Cefazolin, Cefalotin, Cefalexin, Cefaclor, Cefamandole, Cefoxitin, Cefprozil, Cefuroxime, Cefixime, Cefdinir, Cefditoren, Cefoperazone, Cefotaxime, Cefpodoxime, Ceftazidime, Ceftibuten, Ceftizoxime, Ceftriaxone, Cefepime, Ceftaroline fosamil, Ceftobiprole, Teicoplanin, Vancomycin, Telavancin, Clindamycin, Lincomycin, Daptomycin, Azithromycin, Clarithromycin, Dirithromycin, Erythromycin, Roxithromycin, Troleandomycin, Telithromycin, Spiramycin, Aztreonam, Furazolidone, Nitrofurantoin, Linezolid, Posizolid, Radezolid, Torezolid, Amoxicillin, Ampicillin, Azlocillin, Carbenicillin, Cloxacillin, Dicloxacillin, Flucloxacillin, Mezlocillin, Methicillin, Nafcillin, Oxacillin, Penicillin G, Penicillin V, Piperacillin, Penicillin G, Temocillin, Ticarcillin, clavulanate, sulbactam, tazobactam, clavulanate, Bacitracin, Colistin, Polymyxin B, Ciprofloxacin, Enoxacin, Gatifloxacin, Levofloxacin, Lomefloxacin, Moxifloxacin, Nalidixic acid, Norfloxacin, Ofloxacin, Trovafloxacin, Grepafloxacin, Sparfloxacin, Temafloxacin, Mafenide, Sulfacetamide, Sulfadiazine, Silver sulfadiazine, Sulfadimethoxine, Sulfamethizole, Sulfamethoxazole, Sulfanilimide, Sulfasalazine, Sulfisoxazole, Trimethoprim-Sulfamethoxazole, Co-trimoxazole, Sulfonamidochrysoidine, Demeclocycline, Doxycycline, Minocycline, Oxytetracycline, Tetracycline, Clofazimine, Dapsone, Capreomycin, Cycloserine, Ethambutol, Ethionamide, Isoniazid, Pyrazinamide, Rifampicin, Rifabutin, Rifapentine, Streptomycin, Arsphenamine, Chloramphenicol, Fosfomycin, Fusidic acid, Metronidazole, Mupirocin, Platensimycin, Quinupristin, Dalfopristin, Thiamphenicol, Tigecycline, Tinidazole, Trimethoprim, Geneticin, Nourseothricin, Hygromycin, Bleomycin, and Puromycin.

In some embodiments, the dominant selectable marker is a nutritional marker. In some embodiments, the nutritional marker is selected from the group consisting of Phosphite specific oxidoreductase, Alpha-ketoglutarate-dependent hypophosphite dioxygenase, Alkaline phosphatase, Cyanamide hydratase, Melamine deaminase, Cyanurate amidohydrolase, Biuret hydrolyase, Urea amidolyase, Ammelide aminohydrolase, Guanine deaminase, Phosphodiesterase, Phosphotriesterase, Phosphite hydrogenase, Glycerophosphodiesterase, Parathion hydrolyase, Phosphite dehydrogenase, Dibenzothiophene desulfurization enzyme, Aromatic desulfinase, NADH-dependent FMN reductase, Aminopurine transporter, Hydroxylamine oxidoreductaseInvertase, Beta-glucosidase, Alpha-glucosidase, Beta-galactosidase, Alpha-galactosidase, Amylase, Cellulase and Pullulonase.

In some embodiments, the cell is a prokaryotic cell, such as a bacterial cell. In some embodiments, the cell is a eukaryotic cell, such as a mammalian cell, a yeast cell, a filamentous fungi cell, a protist cell, an algae cell, an avian cell, a plant cell or an insect cell.

In some embodiments, the different domains of the dominant selectable marker associate via a protein binding motif. In some embodiments, the protein binding motif is a leucine zipper motif, a Src homology 2 domain, a Src homology 3 domain, a phosphotyrosine binding domain, a LIM domain, a sterile alpha motif domain, a PDZ domain, a FERM domain, a calponin homology domain, a plexkstrin homology domain, a WW domain or a WS×WS motif.

### BRIEF DESCRIPTION OF THE FIGURES

FIG. 1 depicts the result of domain linker prediction for Pseudomonas stutzeri phosphonate dehydrogenase gene ptxD using the DLP-SVM-Joint method.

FIG. 2 depicts the result of domain linker prediction for Pseudomonas stutzeri phosphonate dehydrogenase gene ptxD using the DROP method.

FIG. 3 depicts the aliment of linker regions predicted by two different methods: the DLP-SVM-Joint method (SVM) and the DROP method (DROP) for Pseudomonas stutzeri phosphonate dehydrogenase gene ptxD. The shaded rectangle shows the overlap between two methods. The arrow shows the point of protein split for designing two separately expressed domains.

FIG. 4 is a schematic diagram of post-translational assembly of Pseudomonas stutzeri phosphonate dehydrogenase domains N-pxtD and C-ptxD via coiled-coil interaction between antiparallel leucine linkers fused to the N-pxtD and C-ptxD domains.

FIG. 5 is a map of a ptxD construct used to express Pseudomonas stutzeri phosphonate dehydrogenase gene ptxD in Y. lipolytica strain NS18. Vector ptxD was linearized by PacI/NotI restriction digest before transformation. 2u ori: S. cerevisiae origin of replication; pMB1 ori: E. coli pMB1 origin of replication; AmpR: bla gene used as marker for selection with ampicillin; PR2: Y. lipolytica GPD1 promoter −931 to −1; ptxD: Pseudomonas stutzeri phosphonate dehydrogenase gene ptxD; TER1: Y. lipolytica CYC1 terminator 300 bp after stop; Sc URA3: S. cerevisiae URA3 auxotrophic marker for selection in yeast.

FIG. 6 is a map of a ptxD-split construct used to express and assemble N-pxtD and C-ptxD domains of Pseudomonas stutzeri phosphonate dehydrogenase gene ptxD in Y. lipolytica strain NS18. Vector ptxD-split was linearized by PacI/NotI/PmeI restriction digest before transformation. 2u ori: S. cerevisiae origin of replication; pMB1 ori: E. coli pMB1 origin of replication; AmpR: bla gene used as marker for selection with ampicillin; PR2: Y. lipolytica GPD1 promoter −931 to −1; N-ptxD: N-ter domain of Pseudomonas stutzeri phosphonate dehydrogenase gene ptxD; N-ZIP: leucine zipper for N-ter protein domain; TER1: Y. lipolytica CYC1 terminator 300 bp after stop; PR22: S. cerevisiae TEF1 promoter −412 to −1; C-ZIP: designed leucine zipper for C-ter protein domain; C-ptxD: C-ter domain of Pseudomonas stutzeri phosphonate dehydrogenase gene ptxD; TER2: S. cerevisiae CYC1 terminator 275 bp after stop; Sc URA3: S. cerevisiae URA3 auxotrophic marker for selection in yeast.

FIG. 7 provides the DNA and protein sequences of Pseudomonas stutzeri phosphonate dehydrogenase gene ptxD.

FIG. 8 provides the DNA and protein sequences of the N-terminal and C-terminal domains of Pseudomonas stutzeri phosphonate dehydrogenase gene ptxD.

FIG. 9 provides the DNA and protein sequences of the leucine zippers for the N-terminal and C-terminal domains of Pseudomonas stutzeri phosphonate dehydrogenase gene ptxD.

### DETAILED DESCRIPTION

### Overview

The methods and compositions provided herein (e.g., methods, cells, nucleic acids, polypeptides, proteins and kits) eliminate or significantly reduce the horizontal transfer of genetic elements encoding functional proteins, including but not limited to, genetic elements encoding functional dominant selection markers.

Horizontal gene transfer results in the spread of drug resistance among pathogens. As a consequence, newly discovered and artificial antibiotics can lose their effectiveness over time as bacteria and other organisms develop drug resistance mechanisms, which are then spread to other organisms through horizontal gene transfer.

Since drug resistance and nutritional selection markers are commonly used in the genetic engineering of organisms, transfer of markers from an engineered organism into the environment presents medical and agricultural risks. For example, horizontal transfer of dominant selectable markers increases the likelihood that pathogens acquire drug resistance and thereby become more difficult to treat or prevent disease in humans, livestock and crops. Additionally, the inadvertent spread of markers from genetically engineered to natural organisms also reduces the competitive advantage engineered into genetically modified organisms and, as a result, increases the risk of contamination with undesired native organisms. Thus, horizontal gene transfer can increase the risk of bioreactor contamination with undesired antibiotic resistant microorganisms and crop contamination with herbicide resistant invasive plant species.

Thus, in some embodiments, provided herein are compositions and methods for decreasing the risk of horizontal gene transfer by separately encoding domains of a dominant selectable marker on at least two spatially distinct nucleic acid sequences, where each individual domain alone is not able to function as a selectable marker, but co-expression of the encoded domains results in their association to form a functional selectable marker. Because the individual marker domains are not functional except when assembled with other domains, transfer of individual domains to other organisms through horizontal gene transfer does not confer a selective advantage to the recipient organisms. Thus, the probability that a non-host organism to obtains all of the domains necessary for assembly of a functional dominant selectable marker is significantly lower than the probability of a single horizontal gene transfer event.

### Definitions

The articles “a” and “an” are used herein to refer to one or to more than one (i.e., to at least one) of the grammatical object of the article. By way of example, “an element” means one element or more than one element.

The term “domain” refers to a part of the amino acid sequence of a protein that is able to fold into a stable three dimensional structure independent of the rest of the protein. Natural protein domains are often connected by a domain linker. Methods for identifying domains and domain linkers from a protein's amino acid sequence are known in the art and include, for example, the DLP-SVM-Joint method described in Ebina et al., Biopolymers 92:1-8 (2009), hereby .incorporated by reference in its entirety, and the DROP method, which is described in Ebina et al., Bioinformatics 27:487-494 (2001), hereby incorporated by reference in its entirety.

The term “dominant selectable marker” refers to a selectable marker that permits a cell expressing the selectable marker to grow and/or survive under certain cell culture conditions. A dominant selectable marker can be contrasted with a “negative selectable marker,” which refers to a selectable marker that prevents a cell expressing the selectable marker from growing and/or surviving under certain cell culture conditions. It is possible for a single selectable marker to be both a dominant selectable marker and a negative selectable marker if the selectable marker permits a cell expressing the selectable marker to grow and/or survive under certain cell culture conditions but prevents a cell expressing the selectable marker from growing and/or surviving under other cell culture conditions.

As used herein, the term “nutrient source” includes any material that can be used by a cell to facilitate cell growth and/or survival, including carbon sources.

As used herein, the term “plasmid” refers to a circular DNA molecule that is physically separate from an organism's genomic DNA. Plasmids may be linearized before being introduced into a host cell (referred to herein as a linearized plasmid). Linearized plasmids are not be self-replicating, but may integrate into and be replicated with the genomic DNA of an organism.

The terms “polynucleotide”, and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. A polynucleotide may be further modified, such as by conjugation with a labeling component. In all nucleic acid sequences provided herein, U nucleotides are interchangeable with T nucleotides.

The term “vector” refers to the means by which a nucleic acid can be propagated and/or transferred between organisms, cells, or cellular components. Vectors include plasmids, linear DNA fragments, viruses, bacteriophage, pro-viruses, phagemids, transposons, and artificial chromosomes, and the like, that may or may not be able to replicate autonomously or integrate into a chromosome of a host cell.

### Methods for Reducing Horizontal Gene Transfer of Functional Proteins

In some aspects, provided herein is a method of generating a cell that has a reduced likelihood of horizontally transferring a nucleic acid sequence encoding a functional protein. In some embodiments, this is accomplished by engineering the cell so that the protein is expressed as at least two separate domains encoded by separate expression cassettes. Each of the expressed domains is non-functional when expressed alone, but when the domains are expressed together they associate to form a functional protein. In some embodiments, the protein is a dominant selectable marker.

In some embodiments, the method includes introducing into a cell at least two different polynucleotides encoding different domains of the protein. In some embodiments, the functional protein is divided into 2, 3, 4, 5, 6 or more separate domains. In some embodiments, exactly two different polynucleotides are introduced. In some embodiments, each separate domain is encoded on a separate polynucleotide that is introduced into the cell. In some embodiments, the separate polynucleotides are introduced to the cell simultaneously (e.g., in a single co-transfection). In some embodiments, the separate polynucleotides are introduced into the cell sequentially (e.g., in sequential transfections). Thus, in some embodiments, a single polynucleotide encoding a domain of the protein in introduced into a cell comprising (e.g., in its genome) a nucleic acid sequence encoding a different domain of the protein. In some embodiments, the separate domains are encoded by separate expression cassettes. The separate expression cassettes can be on separate polynucleotides or on a single polynucleotide. In some embodiments, the polynucleotides are plasmids, fragments of plasmids, or linearized plasmids.

In certain embodiments, the methods described herein can be applied to any cell type. In some embodiments, the cell is a prokaryotic cell, such as a bacterial cell. In some embodiments, the cell is a eukaryotic cell, such as a mammalian cell, a yeast cell, a filamentous fungi cell, a protist cell, an algae cell, an avian cell, a plant cell or an insect cell.

In some embodiments, the cell is a microorganism. In some embodiments, the microorganism is a species of the genus Yarrowia, Arxula, Saccharomyces, Ogataea, Pichia, or Escherichia. In some embodiments, the mircororganism is selected from the group consisting of Yarrowia lipolytica, Saccharomyces cerevisiae, Ogataea polymorphs, Pichia pastoris, Arxula adeniovorans, and Escherichia coli.

In certain embodiments, the polynucleotides can be introduced into the cell using any method known in the art. For example, in some embodiments, the polynucleotides are introduced in a vector. One type of vector is a “plasmid”, which refers to a circular double stranded DNA loop into which additional DNA segments may be ligated. In some embodiments, the plasmid is linearized before introduction into the cell. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal eukaryotic vectors). Other vectors (e.g., non-episomal eukaryotic vectors) can be integrated into the genome of a host cell upon introduction into the host cell, and thereby are replicated along with the host genome.

Certain vectors are capable of directing the expression of genes to which they are operatively linked (expression vectors). The expression vectors provided herein are able to facilitate the expression of the encoded domain in a host cell, which means that the expression vectors include one or more regulatory sequences (e.g., promoters, enhancers), selected on the basis of the host cells to be used for expression, which is operatively linked to the nucleic acid sequence to be expressed. The design of the expression vector can depend on such factors as the choice of the host cell to be transformed, the level of expression of protein desired, and the like.

The polynucleotides can be introduced into prokaryotic or eukaryotic host cells via conventional transformation or transfection techniques. Examples of transformation and transfection techniques include calcium phosphate or calcium chloride co-precipitation, DEAE-dextran-mediated transfection, lipofection, electroporation, optical transfection, protoplast fusion, impalefection, hydrodynamic delivery, using a gene gun, magnetofection, and particle bombardment. Polynucleotides can also be introduced by infecting the cells with a viral vector (e.g., an adenovirus vector, an adeno-associated virus vector, a lentivirus vector or a retrovirus vector). Suitable methods for transforming or transfecting host cells can be found in Sambrook et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

In some aspects, provided herein is a kit useful for performing a method described herein. In some embodiments, the kit includes at least two different polynucleotides encoding different domains of a dominant selectable marker. In some embodiments, the different domains of the dominant selectable marker are capable of associating to form a complete dominant selectable marker. In some embodiments, no individual polynucleotide encodes the entire dominant selectable marker. In some embodiments, the polynucleotides are in vectors. In some embodiments, the polynucleotides are plasmids, fragments of plasmids, or linearized plasmids. In some embodiments, the kit comprises exactly two different polynucleotides. In some embodiments, the kit further comprises a cell. In some embodiments, the cell is a eukaryotic cell, such as a mammalian cell, a yeast cell, a filamentous fungi cell, a protist cell, an algae cell, an avian cell, a plant cell or an insect cell. In some embodiments, the kit further comprises a transfection reagent. In some embodiments, the kit further comprises instructions for use.

Cells with Reduced Horizontal Gene Transfer

In certain aspects, provided herein is a cell that has a reduced likelihood of horizontally transferring a protein that it expresses (e.g., a dominant selection marker). In some embodiments, the cell is a prokaryotic cell, such as a bacterial cell. In some embodiments, the cell is a eukaryotic cell, such as a mammalian cell, a yeast cell, a filamentous fungi cell, a protist cell, an algae cell, an avian cell, a plant cell or an insect cell.

In some embodiments, the cell is a microorganism. In some embodiments, the microorganism is a species of the genus Yarrowia, Saccharomyces, Ogataea, Pichia, Arxula or Escherichia. In some embodiments, the mircororganism is selected from the group consisting of Yarrowia lipolytica, Saccharomyces cerevisiae, Ogataea polymorphs, Pichia pastoris, Arxula adeniovorans, and Escherichia coli.

In some embodiments, the cells contains at least two nucleic acid sequences encoding different domains of a protein that are located at spatially distinct positions in the cell. In some embodiments, the different domains of the dominant selectable marker associate to form a functional selectable marker. In some embodiments, no individual nucleic acid sequence encodes the functional dominant selectable marker. In some embodiments, the cell is generated using a method described herein.

In some embodiments, the two nucleic acid sequences are located at different positions in the genome of the cell. In some embodiments, the two nucleic acid sequences are located on different chromosomes. In some embodiments, the two nucleic acid sequences are located on different extrachromosomal elements (e.g., plasmids) within the cell. In some embodiments, one nucleic acid sequence is located in the genomic DNA of the cell, and the other nucleic acid sequence is located on an extrachromosomal element. In some embodiments, at least 10, 20, 30, 40, 50, 60, 70, 80, 90, 100, 150, 200, 250, 300, 350, 400, 450, 500, 600, 700, 800, 900, 1,000, 2,000, 3,000, 4,000, 5,000, 6,000, 7,000, 8,000, 9,000 or 10,000 base-pairs separate the nucleic acid sequences.

### Dominant Selectable Markers

In some embodiments, provided herein are dominant selectable markers that are expressed as separate domains that independently are non-functional but that are capable of associating to form a dominant selectable marker. In some embodiments, provided herein are nucleic acid molecules, cells or organisms encoding such dominant selectable markers and dominant selectable marker domains.

In some embodiments, the dominant selectable marker is a drug resistance marker. A drug resistance marker is a dominant selectable marker that, when expressed by a cell, allows the cell to grow and/or survive in the presence of a drug that would normally inhibit cellular growth and/or survival. Cells expressing a drug resistance marker can be selected by growing the cells in the presence of the drug. In some embodiments, the drug resistance marker is an antibiotic resistance marker. In some embodiments, the drug resistance marker confers resistance to a drug selected from the group consisting of Amphotericin B, Candicidin, Filipin, Hamycin, Natamycin, Nystatin, Rimocidin, Bifonazole, Butoconazole, Clotrimazole, Econazole, Fenticonazole, Isoconazole, Ketoconazole, Luliconazole, Miconazole, Omoconazole, Oxiconazole, Sertaconazole, Sulconazole, Tioconazole, Albaconazole, Fluconazole, Isavuconazole, Itraconazole, Posaconazole, Ravuconazole, Terconazole, Voriconazole, Abafungin, Amorolfin, Butenafine, Naftifine, Terbinafine, Anidulafungin, Caspofungin, Micafungin, Benzoic acid, Ciclopirox, Flucytosine, 5-fluorocytosine, Griseofulvin, Haloprogin, Polygodial, Tolnaftate, Crystal violet, Amikacin, Gentamicin, Kanamycin, Neomycin, Netilmicin, Tobramycin, Paromomycin, Spectinomycin, Geldanamycin, Herbimycin, Rifaximin, Streptomycin, Loracarbef, Ertapenem, Doripenem, Imipenem, Meropenem, Cefadroxil, Cefazolin, Cefalotin, Cefalexin, Cefaclor, Cefamandole, Cefoxitin, Cefprozil, Cefuroxime, Cefixime, Cefdinir, Cefditoren, Cefoperazone, Cefotaxime, Cefpodoxime, Ceftazidime, Ceftibuten, Ceftizoxime, Ceftriaxone, Cefepime, Ceftaroline fosamil, Ceftobiprole, Teicoplanin, Vancomycin, Telavancin, Clindamycin, Lincomycin, Daptomycin, Azithromycin, Clarithromycin, Dirithromycin, Erythromycin, Roxithromycin, Troleandomycin, Telithromycin, Spiramycin, Aztreonam, Furazolidone, Nitrofurantoin, Linezolid, Posizolid, Radezolid, Torezolid, Amoxicillin, Ampicillin, Azlocillin, Carbenicillin, Cloxacillin, Dicloxacillin, Flucloxacillin, Mezlocillin, Methicillin, Nafcillin, Oxacillin, Penicillin G, Penicillin V, Piperacillin, Penicillin G, Temocillin, Ticarcillin, clavulanate, sulbactam, tazobactam, clavulanate, Bacitracin, Colistin, Polymyxin B, Ciprofloxacin, Enoxacin, Gatifloxacin, Levofloxacin, Lomefloxacin, Moxifloxacin, Nalidixic acid, Norfloxacin, Ofloxacin, Trovafloxacin, Grepafloxacin, Sparfloxacin, Temafloxacin, Mafenide, Sulfacetamide, Sulfadiazine, Silver sulfadiazine, Sulfadimethoxine, Sulfamethizole, Sulfamethoxazole, Sulfanilimide, Sulfasalazine, Sulfisoxazole, Trimethoprim-Sulfamethoxazole, Co-trimoxazole, Sulfonamidochrysoidine, Demeclocycline, Doxycycline, Minocycline, Oxytetracycline, Tetracycline, Clofazimine, Dapsone, Capreomycin, Cycloserine, Ethambutol, Ethionamide, Isoniazid, Pyrazinamide, Rifampicin, Rifabutin, Rifapentine, Streptomycin, Arsphenamine, Chloramphenicol, Fosfomycin, Fusidic acid, Metronidazole, Mupirocin, Platensimycin, Quinupristin, Dalfopristin, Thiamphenicol, Tigecycline, Tinidazole, Trimethoprim, Geneticin, Nourseothricin, Hygromycin, Bleomycin, and Puromycin.

In some embodiments, the dominant selectable marker is a nutritional marker. A nutritional marker is a dominant selectable marker that, when expressed by the cell, allows the cell to grow and/or survive using certain nutrient sources. Cells expressing a nutritional marker can be selected by growing the cells under limiting nutrient conditions in which cells expressing the nutritional marker can survive and/or grow, but cells lacking the nutrient marker cannot. In some embodiments, the nutritional marker is selected from the group consisting of Phosphite specific oxidoreductase, Alpha-ketoglutarate-dependent hypophosphite dioxygenase, Alkaline phosphatase, Cyanamide hydratase, Melamine deaminase, Cyanurate amidohydrolase, Biuret hydrolyase, Urea amidolyase, Ammelide aminohydrolase, Guanine deaminase, Phosphodiesterase, Phosphotriesterase, Phosphite hydrogenase, Glycerophosphodiesterase, Parathion hydrolyase, Phosphite dehydrogenase, Dibenzothiophene desulfurization enzyme, Aromatic desulfinase, NADH-dependent FMN reductase, Aminopurine transporter, Hydroxylamine oxidoreductaseInvertase, Beta-glucosidase, Alpha-glucosidase, Beta-galactosidase, Alpha-galactosidase, Amylase, Cellulase and Pullulonase.

In some embodiments, the dominant selectable markers described herein are expressed as separate domains that are nonfunctional alone but that are capable of associating with other domains to form a functional dominant selectable marker. The separate domains of the dominant selectable marker can be identified using methods known in the art. For example, the separate domains can be identified based on a crystal structure of the dominant selectable marker or based on the amino acid sequence of the dominant selectable marker. Methods for identifying domains and domain linkers from a protein's amino acid sequence are known in the art and include, for example, the DLP-SVM-Joint method described in Ebina et al., Biopolymers 92:1-8 (2009), and the DROP method, which is described in Ebina et al., Bioinformatics 27:487-494 (2001). In general, the domains of the dominant selectable marker are devided for separate expression at a position in a domain linker between the domains.

In some embodiments, the domains of the dominant selectable marker are intrinsically able to associate to form a functional dominant selectable marker. In some embodiments, the domains of the dominant selectable marker are engineered to associate via a protein binding motif. In such cases, the domains of the dominant selectable marker are expressed as fusion proteins that include both the dominant selectable marker domain and the protein binding motif. In most cases, the position at which the protein binding motifs are attached to the domain will be selected such that the complex formed following domain association resembles the structure of the native protein. Thus, in some embodiments the protein binding motif is attached to the C-terminus of a domain that is positioned at the N-terminus of the native protein. In some embodiments, the protein binding motif is attached to the N-terminus of a domain that is positioned at the C-terminus of the native protein.

In some embodiments, any protein binding motif can be used to facilitate the association of the dominant selectable marker domains. Examples of protein binding motifs include leucine zippers, a Src homology 2 domain, a Src homology 3 domain, a phosphotyrosine binding domain, a LIM domain, a sterile alpha motif domain, a PDZ domain, a FERM domain, a calponin homology domain, a plexkstrin homology domain, a WW domain or a WS×WS motif.

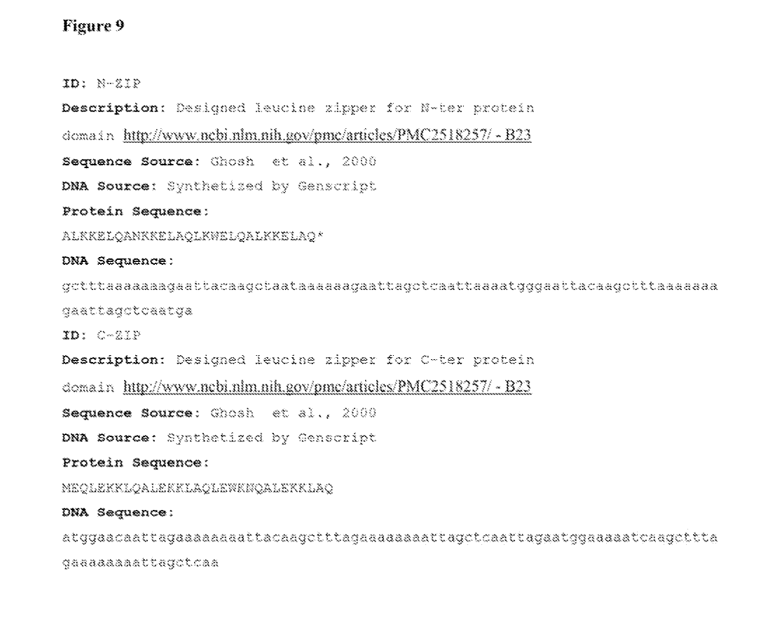

In some embodiments, the protein binding motif is a leucine zipper motif. A leucine zipper is a common three-dimensional protein structural motif that often functions as a dimerization domain. Leucine zipper amino acid sequences are known in the art. For example, exemplary leucine zipper sequences are provided in FIG. 9, and are described in Ghosh et al., J. Am. Chem. Soc. 122:5658 (2000).

In some embodiments, the protein binding motif is a Src homology 2 domain (a SH2 domain). A SH2 domain is a broadly conserved protein domain of about 100 amino acids that allows proteins containing such domains to bind to phosphorylated tyrosine residues on other proteins. SH2 domain sequences are known in that art and are found in many species, including in over 100 human genes. Numerous SH2 domain sequences are known in the art. Examples of human proteins containing SH2 domains include ABL1, ABL2, BCAR3, CHN2, DAPP1, HCK, SLA, SOCS1, SOCS2, SOCS3, TEC, TNS, VAV1, YES1 and ZAP70.

In some embodiments, the protein binding motif is a Src homology 3 domain (a SH3 domain). A SH3 domain is a small, highly conserved protein domain of about 60 amino acids found in about 300 proteins encoded by the human genome. SH3 domains have a characteristic beta-barrel fold that consists of five or six β strands arranged as two tightly packed anti-parallel β sheets. SH3 domains typically bind to proline-rich peptides in a binding partner. The proline-rich peptides bound by SH3 domains often have a consensus sequence of X-P-p-X-P, with X representing aliphatic amino acids, P always representing proline and p sometimes being proline. Examples of human proteins containing SH3 domains include CDC24, CDC25, PI3 kinase, GRB2, SH3D21 and STAC3.

In some embodiments, the protein binding motif is a phosphotyrosine binding domain (a PTB domain). Phosphotyrosine binding domains are protein domains which bind to phosphotyrosin. Examples of phosphotyrosine binding domains are found in the C-terminus of tensin proteins which interacts with the cytoplasmic tail of beta integrin by binding to an NPXY motif. Examples of human proteins containing PTB domains include APBA1, APBA2, EPS8, EPS8L1, TENC1, TNS, DOC1, FRS2, IRS1 and TLN1.

In some embodiments, the protein binding motif is a LIM domain. A LIM domain is a protein structural domain composed of two contiguous zinc finger domains, separated by a two-amino acid residue hydrophobic linker. LIM domains were originally identified in the proteins Lin11, Isl-1 and Mec-3, but have since been identified in many other proteins in both eukaryotes and prokaryotes. The sequence signature of a LIM domain is [C]-[X]₂₋₄-[C]-[X]₁₃₋₁₉-[W]-[H]-[X]₂₋₄-[C]-[F]-[LVI]-[C]-[X]₂₋₄-[C]-[X]₁₃₋₂₀-C-[X]₂₋₄-[C].

In some embodiments, the protein binding motif is a sterile alpha motif domain (a SAM domain). A SAM domain is a protein interaction domain of about 70 amino acids in size found in a wide range of eukaryotic proteins. Sam domains are arranged in a small five-helix bundle with two large interfaces. SAM domains often dimerize with other SAM domains. For example the SAM domain of the fungal protein Ste50p interacts with the SAM domain of Ste11p to facilitate the formation of a heterodimeric complex.

In some embodiments, the protein binding motif is a PDZ domain. A PDZ domain is a common structural domain of 80-90 amino acids found in a wide range of organisms, including bacteria, yeast, plants, viruses and animals. PDZ domains bind to a short region of the C-terminus of other proteins by beta sheet augmentation. There are roughly 260 human PDZ domains, with many proteins containing multiple PDZ domains. Examples of PDZ domain containing human proteins include Erbin, Htra1, Htra2, Htra3, PSD-95, SAP97, CARD11 and PTP-BL. PDZ domains often associate with other protein domains, including SH3 domains.

In some embodiments, the protein binding motif is a FERM domain. A FERM domain is a widespread protein molecule found in numerous cytoskeletal-associated proteins. In most cases, the FERM domain is present at the N-terminus of the FERM domain containing protein. Examples of proteins containing FERM domains include Band 4.1, Ezrin, Moesin, Radixin, Talin, Merlin, NBL4 and TYK2.

In some embodiments, the protein binding motif is a calponin homology domain (a CH domain). CH domains are a family of actin binding domains found in both cytoskeletal proteins and signal transduction proteins. Examples of human proteins containing CH domains include ACTN1, CLMN, DIXDC1, FLNA, IQGAP1, LCP1, MACF1, NAV2, PARVA, SMTN and VAV1. The structure of an exemplary CH domain is provided in Saraste et al., Nat. Struct. Biol. 4:175-179 (1997).

In some embodiments, the protein binding motif is a plexkstrin homology domain (a PH domain). A PH domain is a protein domain of approximately 120 amino acids found in a wide range of proteins. The structure of exemplary PH domains is described in Riddihough Nat. Struct. Biol. 1:755-757 (1994). PH domains have a structure consisting of two perpendicular anti-parallel beta sheets followed by a C-terminal amphipathic helix. Examples of human proteins containing PH domains include ABR, BMK, BTK, DAB2IP, DOK4, EXOC8, GAB1, IRS1, KALRN, NET1, RASA1, ROCK1, RP1 and VEPH1.

In some embodiments, the protein binding motif is a WW domain (also known as the rsp5-domain or the WWP repeating motif). The WW domain is a modular domain of about 40 amino acids that is often repeated up to four times and that interacts with particular proline motifs, including [AP]-[P]-[P]-[AP]-Y motifs. Numerous proteins containing WW from a wide range of species motifs are known in the art, including vertebrate YAP protein, NEDD4 (mouse), RSP5 (yeast), FE65 (rat) and DB10 (tobacco). Detailed information on WW domain interactions is provided in Hu et al., Proteomics 4:643-655 (2004).

In some embodiments, the protein binding motif is a WS×WS motif. The WS×WS motif is a protein interacting motif found in the extracellular domain of certain protein receptors, including type I cytokine receptors. The structure of an exemplary WS×WS motif is provided in Dagil et al., Structure 20:270-282 (2012). Examples of proteins containing a WS×WS motif include the IL-4 receptor, the growth hormone receptor, the prolactin receptor, the erythropoietin receptor the IL-2 receptor, the IL-13 receptor and the IL-6 receptor.

### EXEMPLIFICATION

### Example 1: Identification of Domains in the Nutritional Marker ptxD

To demonstrate that separate expression of marker domains decreases the risk of marker transfer to other organisms, Pseudomonas stutzeri phosphonate dehydrogenase gene ptxD (sequence provided in FIG. 7 based on NCBI Reference Sequence: YP_006457277.1) was selected as an example. When the Pseudomonas stutzeri ptxD gene is expressed in Y. lipolytica it facilitates phosphite consumption by Y. lipolytica and allows its growth under conditions in which phosphite is sole available phosphorous source. Since wild type strains of Y. lipolytica are not able to grow on media in which phosphite is the sole phosphorous source, ptxD functions in Y. lipolytica as a nutritional marker.

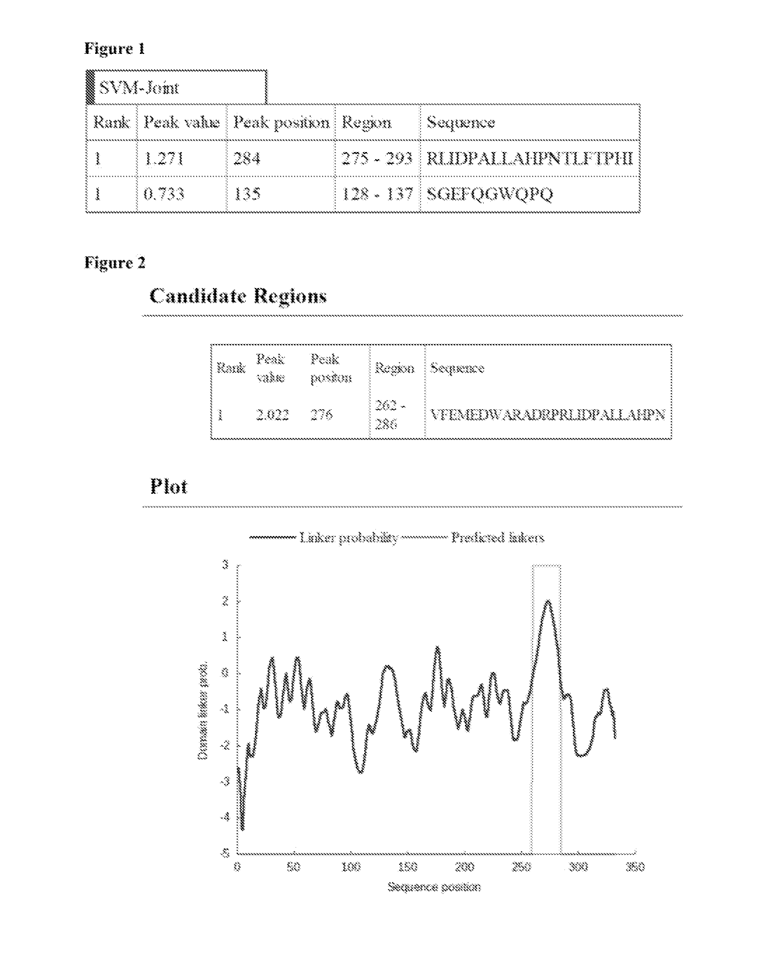

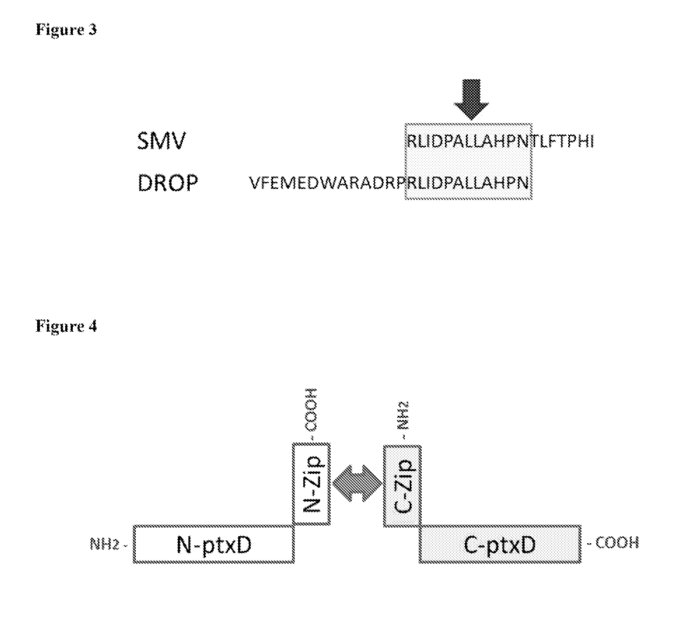

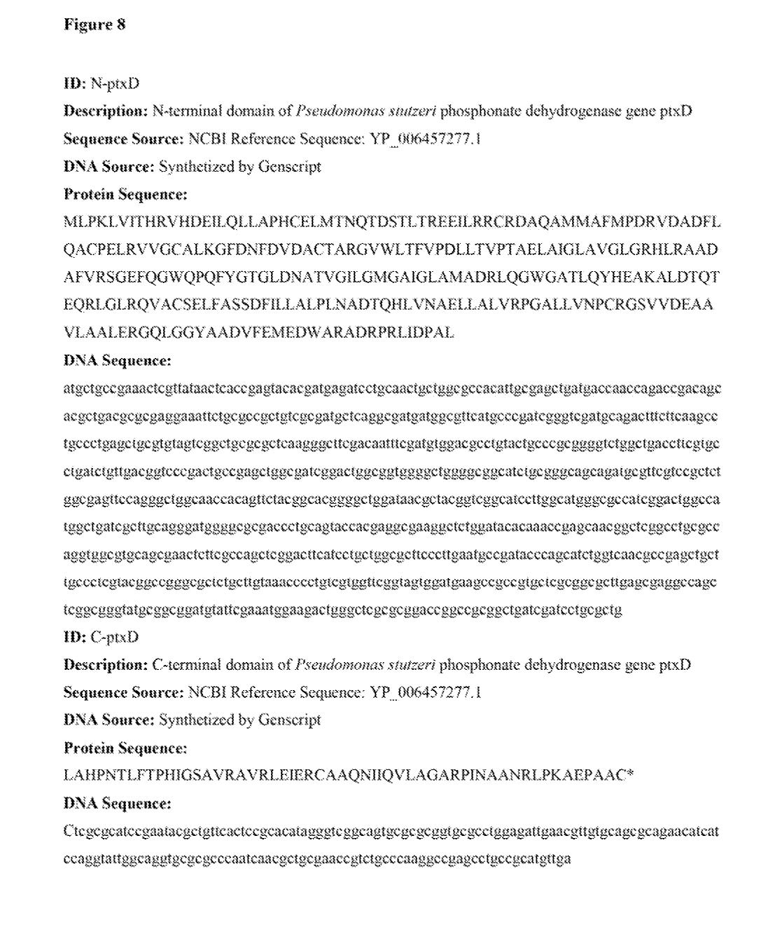

To split the ptxD gene into domains that can fold separately, domain linker sequences was predicted using two methods: DLP-SVM-Joint (Ebina et al., Biopolymers 92:1-8 (2009)) and DROP (Ebina et al., Bioinformatics 27:487-494 (2001)). FIGS. 1 and 2 show the results of these domain linker predictions produced by the DLP-SVM-Joint and DROP methods, respectively. As seen in FIG. 3, the two domain prediction methods identified overlapping linker sequence. A position in the consensus linker sequence was selected as the point at which the N-terminal and C-terminal domains of ptxD would be divided, as indicated by the arrow in FIG. 3. The sequences of the resulting N-terminal and C-terminal domains are provided in FIG. 8.

### Example 2: Creation of Cells Expressing ptxD as Separate N-Terminal and C-Terminal Domains

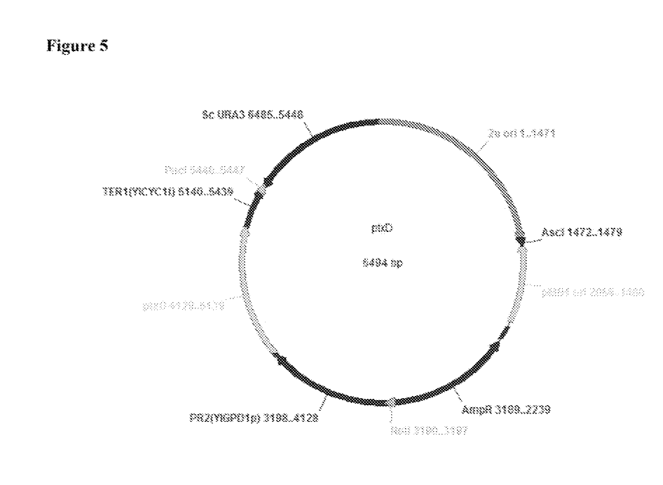

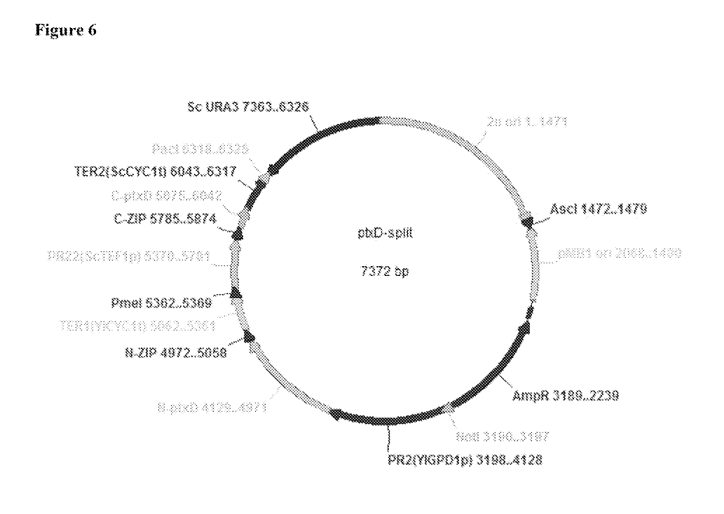

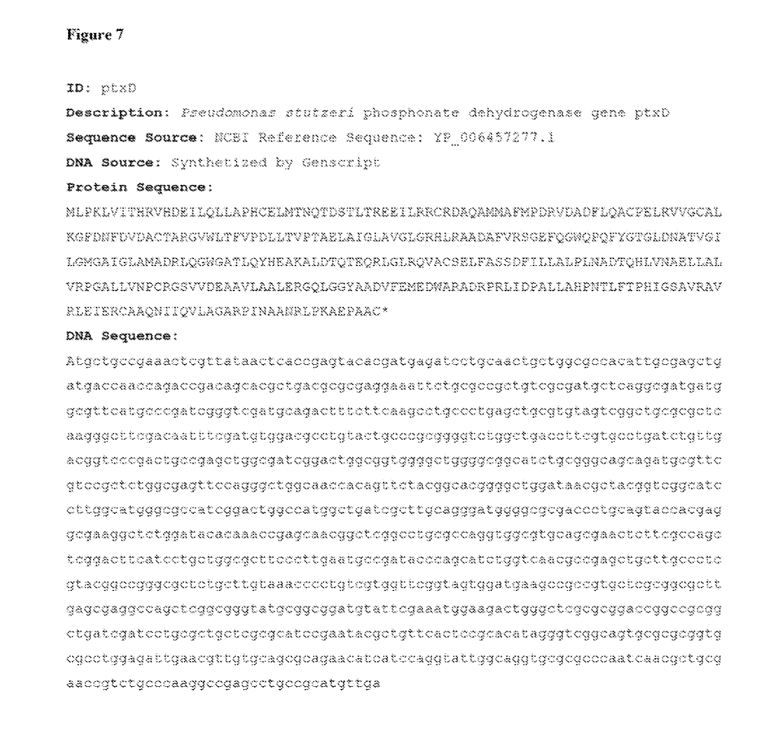

The domains ptxD identified in FIG. 1 are fused to leucine zipper domains (sequences provided in FIG. 9) for post-translational assembly as depicted in FIG. 4. The ptxD is expressed as separate domains or as a single protein for comparison. Y. lipolytica strain NS18 (NRRL YB-392) cells are transformed by one of two expression constructs: ptxD (encoding an entire ptxD protein, depicted in FIG. 5) or ptxD-split (encoding separate ptxD domain/leucine zipper fusion polypeptides, depicted in FIG. 6). Construct ptxD is linearized by PacI/NotI restriction digest before transformation. Construct ptxD-split is linearized by PacI/NotI/PmeI restriction digest before transformation. PacI/NotI/PmeI digest of ptxD-split construct generates two separate DNA fragments, each containing one expression cassette: one encoding an N-terminal domain ptxD/leucine zipper fusion polypeptide and the other encoding a C-terminal domain of ptxD/leucine zipper fusion polypeptide. To obtain separate expression cassettes encoding the individual marker domains, the ptxD-split construct can be digested by PacI/NotI/PmeI endonucleases and the DNA fragments containing individual expression cassettes isolated by using standard gel electrophoresis and gel purification techniques. Alternatively separate expression vectors can be designed and built for each domain of ptxD.

The expression cassettes from the linearized ptxD and ptxD-split plasmids are integrated into the genome of the NS18 strain cells. For linearized ptxD-split, both expression cassettes have to integrate simultaneously into the yeast genome for the ptxD to be functional. As a control experiment each of the ptxD domains are expressed separately in Y. lipolytica cells to demonstrate that each domain is not independently functional.

### Example 3: Reduced Horizontal Gene Transfer in Cells Expressing ptxD as Separate N-Terminal and C-Terminal Domains

The rate of ptxD horizontal transfer to other organisms is determined for the Y. lipolytica strains expressing ptxD as separate domains (NS18+ptxD-split) or as a single polypeptide (NS18+ptxD). Each strain is grown in defined media with phosphite as a phosphorous source in shake flasks in a presence of contaminating organisms that does not have a ptxD gene (e.g., Y. lipolytica strain NS18 that does not express ptxD gene). In order to distinguish contaminating cells from the ptxD expressing strain, the contaminating cells are transformed in advance with antibiotic resistance marker not present in the ptxD expressing strain. The strains are then subjected to serial transfers in which the cell cultures are diluted 100-fold with fresh media every 24-48 hrs. Each time when the cells are diluted, the contaminating cells are added to the flask again. The serial transfers are continued for many generations of cell division (1,000-10,000 or more) and the rate of ptxD transfer is measured by isolating contaminate cells by plating the mixed culture on media containing the contaminant specific antibiotic and measuring what percentage of contaminating cells gained the ability to utilize phosphite. The horizontal gene transfer rate from the NS18+ptxD-split strain is compared to the horizontal gene transfer rate from the NS18+ptxD strain.

As a second method for examining the reduced level of horizontal transfer from the NS18+ptxD-split strain compared to the NS18+ptxD strain, genomic DNA from NS18+ptxD-split and NS18+ptxD strains are isolated and used to transform contaminating organisms such as yeast or bacteria. To increase efficiency of integration of ptxD expressions cassettes into genome of contaminating organism, the genomic DNA of NS18+ptxD-split and NS18+ptxD strains are digested with restriction enzymes. The rate of functional ptxD horizontal transfer is measured by calculating amount of transformants on the plates with defined media and phosphite as a phosphorous source per ug of genomic DNA used for transformation.

### INCORPORATION BY REFERENCE

All of the U.S. patents and U.S. published patent applications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of generating a cell that expresses a functional selectable marker, comprising the step of:
introducing into a cell at least two different polynucleotides encoding different domains of a dominant selectable marker, wherein the different domains of the dominant selectable marker associate to form a functional dominant selectable marker; and no individual polynucleotide encodes the functional dominant selectable marker.

2. The method of claim 1, wherein the dominant selectable marker is a drug resistance marker.

3. The method of claim 2, wherein the drug resistance marker confers resistance to a drug selected from the group consisting of Amphotericin B, Candicidin, Filipin, Hamycin, Natamycin, Nystatin, Rimocidin, Bifonazole, Butoconazole, Clotrimazole, Econazole, Fenticonazole, Isoconazole, Ketoconazole, Luliconazole, Miconazole, Omoconazole, Oxiconazole, Sertaconazole, Sulconazole, Tioconazole, Albaconazole, Fluconazole, Isavuconazole, Itraconazole, Posaconazole, Ravuconazole, Terconazole, Voriconazole, Abafungin, Amorolfin, Butenafine, Naftifine, Terbinafine, Anidulafungin, Caspofungin, Micafungin, Benzoic acid, Ciclopirox, Flucytosine, 5-fluorocytosine, Griseofulvin, Haloprogin, Polygodial, Tolnaftate, Crystal violet, Amikacin, Gentamicin, Kanamycin, Neomycin, Netilmicin, Tobramycin, Paromomycin, Spectinomycin, Geldanamycin, Herbimycin, Rifaximin, Streptomycin, Loracarbef, Ertapenem, Doripenem, Imipenem, Meropenem, Cefadroxil, Cefazolin, Cefalotin, Cefalexin, Cefaclor, Cefamandole, Cefoxitin, Cefprozil, Cefuroxime, Cefixime, Cefdinir, Cefditoren, Cefoperazone, Cefotaxime, Cefpodoxime, Ceftazidime, Ceftibuten, Ceftizoxime, Ceftriaxone, Cefepime, Ceftaroline fosamil, Ceftobiprole, Teicoplanin, Vancomycin, Telavancin, Clindamycin, Lincomycin, Daptomycin, Azithromycin, Clarithromycin, Dirithromycin, Erythromycin, Roxithromycin, Troleandomycin, Telithromycin, Spiramycin, Aztreonam, Furazolidone, Nitrofurantoin, Linezolid, Posizolid, Radezolid, Torezolid, Amoxicillin, Ampicillin, Azlocillin, Carbenicillin, Cloxacillin, Dicloxacillin, Flucloxacillin, Mezlocillin, Methicillin, Nafcillin, Oxacillin, Penicillin G, Penicillin V, Piperacillin, Penicillin G, Temocillin, Ticarcillin, clavulanate, sulbactam, tazobactam, clavulanate, Bacitracin, Colistin, Polymyxin B, Ciprofloxacin, Enoxacin, Gatifloxacin, Levofloxacin, Lomefloxacin, Moxifloxacin, Nalidixic acid, Norfloxacin, Ofloxacin, Trovafloxacin, Grepafloxacin, Sparfloxacin, Temafloxacin, Mafenide, Sulfacetamide, Sulfadiazine, Silver sulfadiazine, Sulfadimethoxine, Sulfamethizole, Sulfamethoxazole, Sulfanilimide, Sulfasalazine, Sulfisoxazole, Trimethoprim-Sulfamethoxazole, Co-trimoxazole, Sulfonamidochrysoidine, Demeclocycline, Doxycycline, Minocycline, Oxytetracycline, Tetracycline, Clofazimine, Dapsone, Capreomycin, Cycloserine, Ethambutol, Ethionamide, Isoniazid, Pyrazinamide, Rifampicin, Rifabutin, Rifapentine, Streptomycin, Arsphenamine, Chloramphenicol, Fosfomycin, Fusidic acid, Metronidazole, Mupirocin, Platensimycin, Quinupristin, Dalfopristin, Thiamphenicol, Tigecycline, Tinidazole, Trimethoprim, Geneticin, Nourseothricin, Hygromycin, Bleomycin, and Puromycin.

4. The method of claim 1, wherein the dominant selectable marker is a nutritional marker.

5. The method of claim 4, wherein the nutritional marker is selected from the group consisting of Phosphite specific oxidoreductase, Alpha-ketoglutarate-dependent hypophosphite dioxygenase, Alkaline phosphatase, Cyanamide hydratase, Melamine deaminase, Cyanurate amidohydrolase, Biuret hydrolyase, Urea amidolyase, Ammelide aminohydrolase, Guanine deaminase, Phosphodiesterase, Phosphotriesterase, Phosphite hydrogenase, Glycerophosphodiesterase, Parathion hydrolyase, Phosphite dehydrogenase, Dibenzothiophene desulfurization enzyme, Aromatic desulfinase, NADH-dependent FMN reductase, Aminopurine transporter, Hydroxylamine oxidoreductaseInvertase, Beta-glucosidase, Alpha-glucosidase, Beta-galactosidase, Alpha-galactosidase, Amylase, Cellulase and Pullulonase.

6. The method of claim 1, wherein the cell is a prokaryotic cell.

7. The method of claim 6, wherein the cell is a bacterial cell.

8. The method of claim 1, wherein the cell is a eukaryotic cell.

9. The method of claim 8, wherein the cell is a mammalian cell, a yeast cell, a filamentous fungi cell, a protist cell, an algae cell, an avian cell, a plant cell or an insect cell.

10. The method of claim 1, wherein the different domains of the dominant selectable marker associate via a protein binding motif.

11. The method of claim 10, wherein the protein binding motif is a leucine zipper motif, a Src homology 2 domain, a Src homology 3 domain, a phosphotyrosine binding domain, a LIM domain, a sterile alpha motif domain, a PDZ domain, a FERM domain, a calponin homology domain, a plexkstrin homology domain, a WW domain or a WS×WS motif.

12. The method of claim 1, wherein the polynucleotides are introduced to the cell in vectors.

13. The method of claim 1, wherein the polynucleotides are plasmids, fragments of plasmids, or linearized plasmids.

14. The method of claim 1, wherein two different polynucleotides are introduced.

15. The method of claim 1, wherein the different polynucleotides integrate into different positions in the genome of the cell.

16. A method of generating a cell that expresses a functional dominant selectable marker, comprising the step of introducing into a cell that expresses a first domain of a dominant selectable marker, a polynucleotide encoding a second domain of the dominant selectable marker, wherein the first domain and the second domain associate to form a functional dominant selectable marker; the polynucleotide does not encode the functional dominant selectable marker; and the cell did not express the functional dominant selectable marker before transfection.

17-29. (canceled)

30. A cell, comprising at least two nucleic acid sequences located at different positions in the genome of the cell and encoding different domains of a dominant selectable marker, wherein the different domains of the dominant selectable marker associate to form a functional selectable marker; and no individual nucleic acid sequence encodes the functional dominant selectable marker.

31-41. (canceled)

42. A kit, comprising at least two different polynucleotides encoding different domains of a dominant selectable marker, wherein the different domains of the dominant selectable marker are capable of associating to form a complete dominant selectable marker; and no individual polynucleotide encodes the entire dominant selectable marker.

43-51. (canceled)
